(19) United States
(12) Patent Application Publication
(10) Pub. No.: US 2025/0198983 A1
(43) Pub. Date: Jun. 19, 2025

(54) METHOD AND APPARATUS WITH CELLULAR ELECTRICAL SIGNAL MEASURING

(71) Applicant: Samsung Electronics Co., Ltd., Suwon-si (KR)

(72) Inventors: Jaechun LEE, Suwon-si (KR); Chisung BAE, Suwon-si (KR); Kitae PARK, Suwon-si (KR)

(73) Assignee: Samsung Electronics Co., Ltd., Suwon-si (KR)

(21) Appl. No.: 18/919,760

(22) Filed: Oct. 18, 2024

(30) Foreign Application Priority Data
Dec. 18, 2023 (KR) 10-2023-0184818

(51) Int. Cl. G01N 33/483; C12M 1/34; C12M 1/42

(52) U.S. Cl. CPC G01N 33/4836; C12M 35/02; C12M 41/46

(57) ABSTRACT

A device including a plurality of electrodes, a plurality of electric current generators, the plurality of electric current generators connected to each of the plurality of electrodes and configured to apply an electric current to a cell cultured on an electrode, a plurality of voltage sensors, the plurality of voltage sensors being electrically and respectively connected to each of the plurality of electrodes and configured to measure a potential of the cell cultured on the corresponding electrode, a processor, and a memory, the processor being configured to determine a required time for electroporation to occur in a target cell based on a potential of the target cell that occurs in response to an electric current being applied to the target cell cultured on a target electrode among the plurality of electrodes and update a magnitude of an updated electric current to be applied to the target cell based on the required time.

### CROSS-REFERENCE TO RELATED APPLICATIONS

This application claims the benefit under 35 USC § 119(a) of Korean Patent Application No. 10-2023-0184818, filed on Dec. 18, 2023, in the Korean Intellectual Property Office, the entire disclosure of which is incorporated herein by reference for all purposes.

### BACKGROUND

### 1. Field

The following description relates to a method and apparatus with cellular electrical signal measuring.

### 2. Description of Related Art

A variety of methods may be used to analyze a human biometric signal. For example, signals from neurons measured by a plurality of electrodes may be used to analyze brain waves. In addition, the manner of transmitting signals of a brain may be interpreted by passing a certain amount of electric current through nerve cells of the brain and observing intracellular signals. Typically, a microelectrode array may include a plurality of electrodes, each of which may measure an electrical signal generated at the cellular level.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In a general aspect, here is a device including a plurality of electrodes in contact with a medium for culturing cells, a plurality of electric current generators, the plurality of electric current generators being electrically and respectively connected to each of the plurality of electrodes and configured to apply an electric current to a cell cultured on a corresponding electrode, a plurality of voltage sensors, the plurality of voltage sensors being electrically and respectively connected to each of the plurality of electrodes and configured to measure a potential of the cell cultured on the corresponding electrode, and a processor configured to execute instructions, and a memory storing the instructions, an execution of the instructions configures the processor to determine a required time for electroporation to occur in a target cell based on a potential of the target cell that occurs in response to an electric current being applied to the target cell cultured on a target electrode among the plurality of electrodes and update a magnitude of an updated electric current to be applied to the target cell based on the required time.

The processor may be further configured to determine a time taken for a magnitude of the potential of the target cell, in response to the electric current being applied to the target cell, to reach a preset threshold potential to be the required time.

The processor may be further configured to in response to the required time being greater than a reference time for which the electroporation is intended to occur, increase the magnitude of the updated electric current and, in response to the required time being less than the reference time, decrease the magnitude of the updated electric current.

The processor may be further configured to determine the required time for electroporation to occur in the target cell while increasing the magnitude of the electric current being applied to the target cell and determine a rate of increasing the updated electric current based on the required time.

The processor may be further configured to determine the rate of increasing the updated electric current according to an amount of electric charge being applied to the target cell during a reference time for which the electroporation is intended to occur.

The processor may be further configured to determine the required time for electroporation to occur in the target cell when the electric current being applied to the target cell is based on a direct current (DC) voltage being applied to the target electrode.

The processor may be further configured to, in response to a difference between the DC voltage of the target cell and a reference voltage being greater than or equal to a preset threshold voltage, determine that electroporation has occurred in the target cell cultured on the target electrode, the DC voltage may be based on the electric current being applied to the target electrode, and the reference voltage may be based on an electric current being applied to a target electrode on which a cell is not cultured.

The processor may be further configured to perform intracellular recording on the cells in response to determining that electroporation has occurred in the cells for each of the plurality of electrodes.

In a general aspect, here is provided an operating method of a device including applying an electric current to a target cell cultured on a target electrode among a plurality of electrodes in contact with a medium for culturing cells, determining a required time for electroporation to occur in the target cell based on a potential of the target cell that occurs in response to the electric current being applied to the target electrode, and updating, based on the required time, a magnitude of an updated electric current to be applied to the target cell.

The determining of the required time may include determining a time taken for a magnitude of the potential of the target cell to reach a preset threshold potential to be the required time in response to the electric current being applied to the target cell.

The updating of the magnitude of the electric current may include increasing the magnitude of the updated electric current in response to the required time being greater than a reference time for which the electroporation is intended to occur and decreasing the magnitude of the updated electric current in response to the required time being less than the reference time.

The determining of the required time may include determining the required time for electroporation to occur in the target cell while increasing the magnitude of the electric current being applied to the target cell and the updating of the magnitude of the electric current may include determining an increase rate of the updated electric current based on the required time.

The updating of the magnitude of the electric current may include determining the increase rate of the updated electric current according to an amount of electric charge applied to the target cell during a reference time for which the electroporation is intended to occur.

The determining of the required time may include determining the required time for electroporation to occur in the target cell when the electric current being applied to the target cell is based on a direct current (DC) voltage being applied to the target electrode.

The determining of the required time may include determining, in response to a difference between the DC voltage of the target cell and a reference voltage being greater than or equal to a preset threshold voltage, that electroporation has occurred in the target cell cultured on the target electrode and determining a required time taken until electroporation has occurred in the target cell, the DC voltage may be based on the electric current being applied to the target electrode, and the reference voltage may be based on an electric current being applied to the target electrode on which a cell is not cultured.

The method may include performing intracellular recording on the cells in response to determining that electroporation has occurred in the cells for each of the plurality of electrodes.

In a general aspect, here is provided a non-transitory computer-readable storage medium storing instructions that, in response to being executed by a processor, cause the processor to perform the method.

In a general aspect, here is provided an apparatus including a processor configured to execute instructions and a memory storing the instructions, wherein execution of the instructions configures the processor to measure a potential of a respective cell cultured on a respective electrode, of a plurality of electrodes, by a respective voltage sensor, of a plurality of voltage sensors, occurring in response to an electric current being applied to the respective cell by the respective electrode, the current being by generated by a respective electric current generator, of a plurality of electric current generators, determine a required time for electroporation to occur in the respective cell based on the potential of the respective cell, and update a magnitude of the electric current being applied to the target cell based on the required time.

The processor may be further configured to synchronize respective times of electroporation among plural cells including the respective cell by adjusting respective plural magnitudes of respective plural electric currents being applied to the plural cells.

The adjusting may include increasing a respective plural magnitude in response to a respective required time for a cell among the plural cells being greater than a reference time for which the electroporation is intended to occur and decreasing the respective plural magnitude in response to the respective required time being less than the reference time.

### BRIEF DESCRIPTION OF THE DRAWINGS

FIG. 1 illustrates an example device for measuring an electrical signal of a cell according to one or more embodiments.

FIG. 2 illustrates an example operation of measuring a potential of a cell when an electric current is applied to the cell according to one or more embodiments.

FIG. 3 illustrates an example operation of determining a time required for electroporation to occur according to one or more embodiments.

FIG. 4 illustrates an example operation of the first electroporation according to one or more embodiments.

FIGS. 5 and 6 illustrate examples of times required for electroporation to occur according to the location of cells cultured on an electrode according to one or more embodiments.

FIG. 7 illustrates an example operation of subsequent electroporation according to one or more embodiments.

FIG. 8 illustrates an example operation of generating electroporation by increasing a magnitude of an electric current applied to a cell and of performing intracellular recording according to one or more embodiments.

FIG. 9 illustrates an example operation of generating electroporation by applying an electric current and of performing intracellular recording according to one or more embodiments.

FIGS. 10 to 12 illustrate example operations of determining a time required for electroporation to occur, based on a direct current (DC) voltage of a cell, according to one or more embodiments.

FIG. 13 illustrates an example electronic device for measuring an electrical signal of a cell according to one or more embodiments.

FIG. 14 illustrates an example method of measuring an electrical signal of a cell according to one or more embodiments.

Throughout the drawings and the detailed description, unless otherwise described or provided, the same drawing reference numerals may be understood to refer to the same or like elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences within and/or of operations described herein are merely examples, and are not limited to those set forth herein, but may be changed as will be apparent after an understanding of the disclosure of this application, except for sequences within and/or of operations necessarily occurring in a certain order. As another example, the sequences of and/or within operations may be performed in parallel, except for at least a portion of sequences of and/or within operations necessarily occurring in an order, e.g., a certain order. Also, descriptions of features that are known after an understanding of the disclosure of this application may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Throughout the specification, when a component or element is described as being “on”, “connected to,” “coupled to,” or “joined to” another component, element, or layer it may be directly (e.g., in contact with the other component or element) “on”, “connected to,” “coupled to,” or “joined to” the other component, element, or layer or there may reasonably be one or more other components, elements, layers intervening therebetween. When a component or element is described as being “directly on”, “directly connected to,” “directly coupled to,” or “directly joined” to another component or element, there can be no other elements intervening therebetween. Likewise, expressions, for example, “between” and “immediately between” and “adjacent to” and “immediately adjacent to” may also be construed as described in the foregoing.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

The terminology used herein is for describing various examples only and is not to be used to limit the disclosure. The articles “a,” “an,” and “the” are intended to include the plural forms as well, unless the context clearly indicates otherwise. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof, or the alternate presence of an alternative stated features, numbers, operations, members, elements, and/or combinations thereof. Additionally, while one embodiment may set forth such terms “comprise” or “comprises,” “include” or “includes,” and “have” or “has” specify the presence of stated features, numbers, operations, members, elements, and/or combinations thereof, other embodiments may exist where one or more of the stated features, numbers, operations, members, elements, and/or combinations thereof are not present.

As used herein, the term “and/or” includes any one and any combination of any two or more of the associated listed items. The phrases “at least one of A, B, and C”, “at least one of A, B, or C”, and the like are intended to have disjunctive meanings, and these phrases “at least one of A, B, and C”, “at least one of A, B, or C”, and the like also include examples where there may be one or more of each of A, B, and/or C (e.g., any combination of one or more of each of A, B, and C), unless the corresponding description and embodiment necessitates such listings (e.g., “at least one of A, B, and C”) to be interpreted to have a conjunctive meaning.

Unless otherwise defined, all terms, including technical and scientific terms, used herein have the same meaning as commonly understood by one of ordinary skill in the art to which this disclosure pertains and based on an understanding of the disclosure of the present application. Terms, such as those defined in commonly used dictionaries, are to be interpreted as having a meaning that is consistent with their meaning in the context of the relevant art and the disclosure of the present application and are not to be interpreted in an idealized or overly formal sense unless expressly so defined herein. The use of the term “may” herein with respect to an example or embodiment, e.g., as to what an example or embodiment may include or implement, means that at least one example or embodiment exists where such a feature is included or implemented, while all examples are not limited thereto.

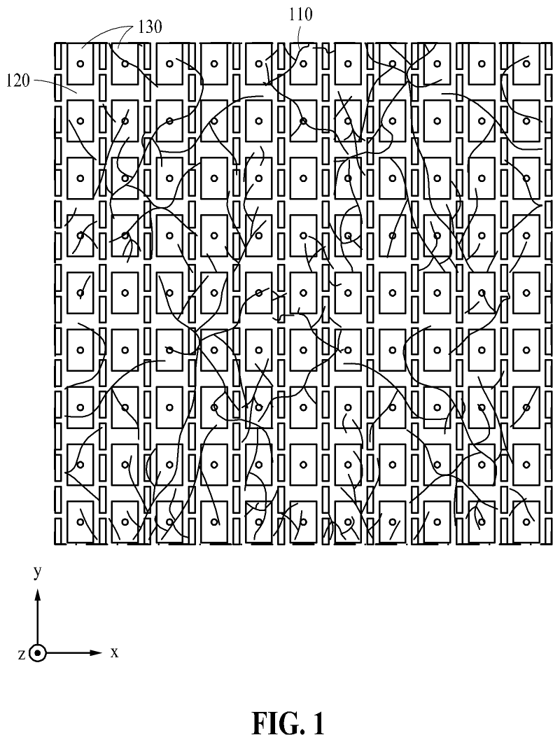

FIG. 1 illustrates an example device for measuring an electrical signal of a cell according to one or more embodiments.

Referring to FIG. 1, in a non-limiting example, a device for measuring an electrical signal of a cell 100 (i.e., a device or the device 100) is illustrated provided alongside an illustration of a cell 110. In an example, the device 100 may detect an electrical signal generated by the cell 110. For example, the cell 110 may be a neuron but is not limited thereto. A plurality of cells may send and receive an electrical signal between each other. In an example, the device may be provided immersed in an aqueous solution. The electrical signal generated by the cell 110 may be transmitted to an electrode through the aqueous solution.

In an example, the device 100 may include a system base 120 and an electrode 130 for receiving the electrical signal of the cell 110. In an example, there may be one or more electrodes 130. The electrode 130 may sit on the system base 120. The system base 120 may include a circuit that collects and analyzes signals received by the electrode 130. The system base 120 and electrode 130 may be electrically and physically connected.

In an example, one or more electrodes 130 may be arranged on a planar surface. The one or more electrodes may be provided in a horizontal and vertical alignment. The one or more electrodes may support the plurality of cells and may each receive an electrical signal generated by the cells. The one or more electrodes may be disposed side by side on an XY plane. The electrical signal generated by the cell 110 may move in the Z-axis direction through the aqueous solution and be transmitted to the inside of the electrode 130. The one or more cells may be cultured, or mounted, on the plurality of electrodes so that an electrical signal generated at the cellular level may be received through the electrodes (i.e., sensed by the circuit).

In an example, the one or more electrodes may be for stimulating and sensing cells and may be used to sense an electrical signal at the cellular level. The device may measure a state of a cell through the contact impedance that occurs between the electrodes and the cells.

The one or more electrodes described above may be implemented as microelectrode array (MEA). For example, the MEA may include tens of thousands of channels within a chip to analyze connections between neurons or brain cells at the network level.

In an example, a medium for culturing a cell may include a nutrient source in the form of a liquid or a gel conceived to proliferate a microorganism, a cell, and the like. The medium may also be referred to as a ‘culture soil’ or a ‘culture medium’. The medium may be contained in a cell culture chamber or a cell culture vessel. The medium may be, for example, a saline solution, but is not limited thereto.

In an example, the device may be used for a drug screening system to test the efficacy or toxicity of drugs or for extracting memory information from neurons by sensing an electrical signal at the cellular level and analyzing a response status of a cell to drugs or a mechanism of neuronal operation.

For the analysis of a large-scale biological neural network, the device may sense an electrical signal from neurons using the MEA and may obtain information on a relationship between cells. The device may perform intracellular recording, which is an operation of a highly sensitive sensing of an intracellular potential through an electroporation technique in which an electric current is applied to an electrode to form a hole in a membrane of a cell adjacent to the electrode.

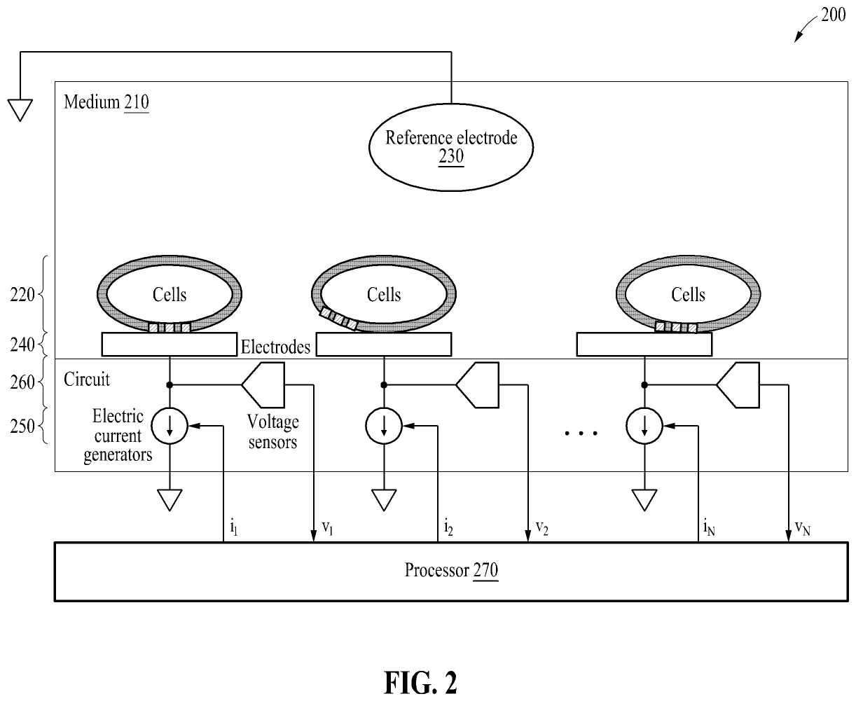

FIG. 2 illustrates an example operation of measuring a potential of a cell when an electric current is applied to the cell according to one or more embodiments.

Referring to FIG. 2, in a non-limiting example, a device 200, such as an example of device 100 of FIG. 1, may include a reference electrode 230, a plurality of electrodes 240, a plurality of electric current generators 250, a plurality of voltage sensors 260, and a processor 270. Cells 220 may be cultured in a medium 210, and the plurality of electrodes 240 may come into contact with the medium 210 for culturing the cells 220.

A cell may be cultured on each of the plurality of electrodes 240, and a location or shape of the cell cultured on each electrode may vary. For example, a cell may be cultured on an electrode in close contact, whereas a cell may not be in close contact with another electrode but be cultured spaced apart by a certain size or at an angle on the another electrode.

In an example, of electroporation, which forms a hole in a cell membrane, may be implemented by applying an electric current to a cell through an electrode on which the cell is cultured. To this end, an electric current generator for applying an electric current to the electrode and a voltage sensor for measuring a voltage formed on the electrode may be disposed on each electrode. When an electric current generator applies an electric current to a corresponding electrode, all or a portion of the applied electric current may flow through a cell cultured on the electrode. Thus, for ease of description, the expression “applying an electric current to an electrode” and the expression “applying an electric current to a cell” may be interchangeably used herein. In addition, since it is self-evident that electroporation occurs in a cell due to an electric current applied to the cell, the expression “electroporation occurs in a cell” and the expression “electroporation occurs in an electrode” may be interchangeably used herein for ease of description.

In an example, each voltage sensor may include an analog front-end (AFE). The AFE may stimulate a cell that comes into contact with or is adjacent to a corresponding electrode and may record an excitation of the cell by stimulation in the form of an electrical signal. The AFE may be a block for digitizing an analog signal in a digital system. The digital system may be configured for processing this analog signal into a digital signal and may include or be a circuit in which an analog preposition circuit and an analog-to-digital converter (ADC) are integrated, for example, in a single chip. The AFE may include, for example, a stimulator for stimulating a cell, a signal amplifier that amplifies a signal detected in cells, and an ADC that converts an amplified (analog) signal to a digital signal, but is not limited thereto. In another example, the AFE may include an instrumentation amplifier (IA) and an ADC corresponding to each electrode. Some AFEs may amplify an electrical signal measured by each electrode through a corresponding IA and may convert the amplified electrical signal to a digital signal through a corresponding ADC.

In an example, the reference electrode 230 may be connected to a ground and disposed in the medium 210. When an electric current is applied by an electric current generator corresponding to each of the plurality of electrodes 240, the electric current generated by the electric current generator may flow along a closed loop formed by the reference electrode 230, each electrode, and each electric current generator.

In an example, an operation of the plurality of electric current generators 250 may be controlled by the processor 270. In addition, information on a voltage sensed by each of the plurality of voltage sensors 260 may be transmitted to the processor 270. The processor 270 may function as both a controller that controls the plurality of electric current generators 250 and a processor that processes information on the sensed voltage, in an example, but may be referred to as a processor herein for ease of description.

A magnitude of an electric current and a time needed to create electroporation may vary depending on a location of a cultured cell on each electrode. Thus, when an electric current of a same magnitude is applied to the plurality of electrodes 240, electroporation may occur later in cells cultured on some electrodes than in other cells cultured on other electrodes. In addition, when an electric current continues to be applied to the cells in which its respective electroporation occurred quickly, damage to the cell may occur. Thus, a magnitude of an electric current applied to each electrode may be controlled so that electroporation may occur simultaneously in the cells 220 cultured on the plurality of electrodes 240. Intracellular recording may be performed after electroporation occurs in the cells 220 cultured on the plurality of electrodes 240.

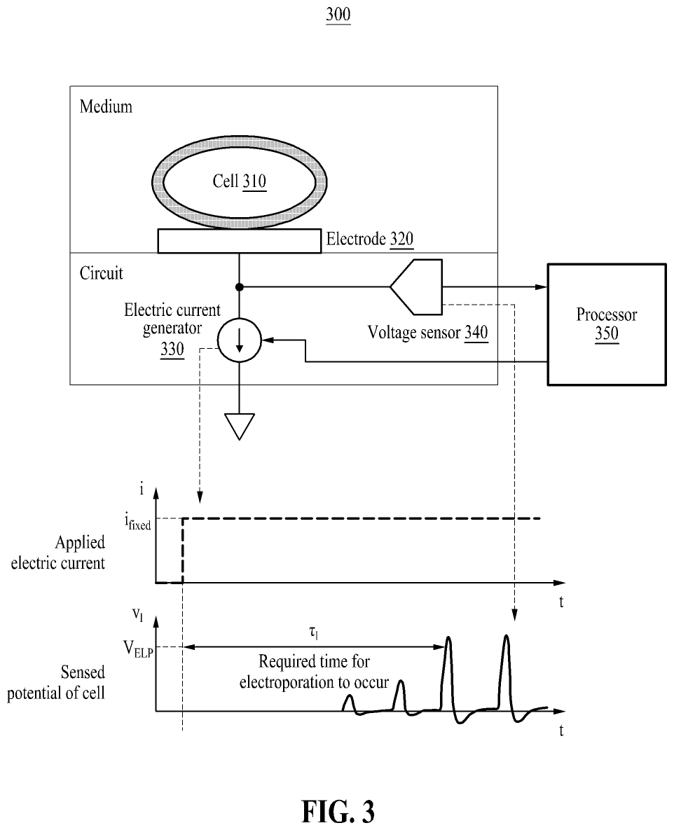

FIG. 3 illustrates an example operation of determining a time required for electroporation to occur according to one or more embodiments.

Referring to FIG. 3, in a non-limiting example, device 300 may include an electronic current generator 330. The electronic current generator may include an electrode 320 through which an electronic current may be applied to cell 310 and a voltage sensor 340. In an example of an operation of the device 300, an amount of time required to for electroporation to occur after applying an electric current to an electrode 320 is determined.

In an operation of the device 300, when an electric current generator 330 applies an electric current i_{fixed} of a preset magnitude to the electrode 320, the electric current i_{fixed} may be applied to a cell 310 through the electrode 320. In an example, the voltage sensor 340 may measure a potential of the cell 310. When a certain period of time elapses after an electric current is applied, the potential of the cell 310 being sensed by the voltage sensor 340 may change. When the potential of the cell 310 reaches a predetermined threshold potential v_{ELP}, a processor 350 may determine that electroporation has occurred in the cell 310. A time required for electroporation to occur in the cell 310 may be determined according to the difference between a timepoint of an onset of electric current application and a timepoint at which the potential of the cell 310 reaches the threshold potential v_{ELP}. Here, the potential of the cell 310 may represent an alternating current (AC) component value, that is, a component value that changes rapidly over time. ELP in v_{ELP} may denote ELectroPoration.

In an example, the threshold potential v_{ELP}, which is a criterion for determining whether electroporation has occurred in the cell 310, may be determined as described below. The threshold potential v_{ELP} may be determined based on distribution data of a signal size obtained by performing extracellular recording before electroporation occurred and a signal size obtained by performing intracellular recording after electroporation occurred. For example, when a signal size of extracellular recording is confirmed to be about 50˜150 μV and a signal size of intracellular recording is confirmed to have a distribution greater than 500 μV after repeated experiments, the threshold potential v_{ELP} may be determined to be 200 μV. Alternatively, when a signal size of extracellular recording is confirmed to be about 50˜300 μV and a signal size of intracellular recording is confirmed to have a distribution greater than 2 mV after repeated experiments, the threshold potential v_{ELP} may be determined to be 1 mV.

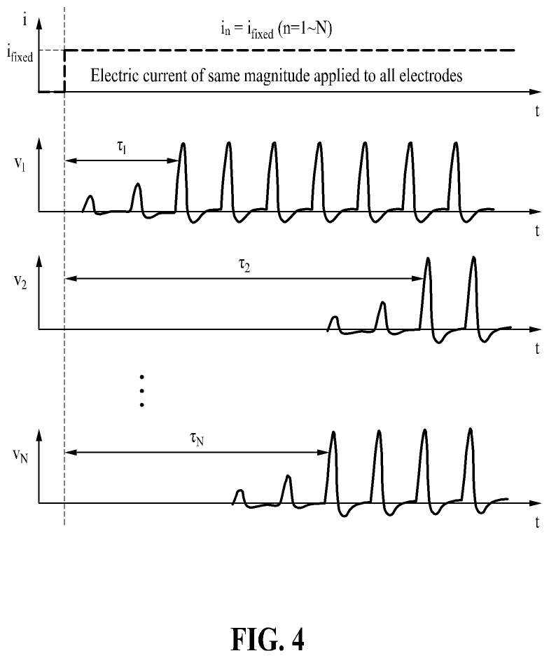

FIG. 4 illustrates an example operation of the first electroporation according to one or more embodiments.

Referring to FIG. 4, a diagram illustrating potentials received by each of a plurality of electrodes describes variations in the amount of time required to for electroporation to occur when an electric current to generate the first electroporation, of a same magnitude, is applied to different electrodes of the plurality of electrodes.

An operation of performing intracellular recording after electroporation may be repeated multiple times. In order to minimize cell damage by synchronizing the time of an occurrence of electroporation in a plurality of cells as much as possible, the magnitude of an electric current applied to each electrode may be updated each time intracellular recording is performed after electroporation.

One reason that there may be different times required for electroporation to occur in the plurality of electrodes may be because a location of each cultured cell may be different for each electrode. The time required for electroporation to occur may tend to be inversely proportional to a voltage applied to a cell membrane. An electric current that forms the voltage in the cell membrane may be selectively output differently for each electrode. Even when electric currents having the same magnitude are applied to the plurality of electrodes, a location of adjacent cells may not be the same for each electrode, and a leakage current that is generated between a particular electrode and cell may be different depending on a distance between the electrode and the cell and/or an arrangement structure. A valid electric current, which is obtained by subtracting a leakage current from an applied current, may reach a cell membrane and form a voltage. When valid electric currents having smaller magnitudes than an applied current reach a cell membrane, magnitudes of the voltages formed in a cell membrane may vary. Accordingly, the time required for electroporation to occur may also vary depending on the change in the magnitude of a voltage formed in a cell membrane.

In order to synchronize the time of the occurrence of electroporation in a plurality of electrodes, an appropriate magnitude of an electric current needed to compensate for a leakage current for each electrode may have to be found. When a large electric current is applied to an electrode in order to generate electroporation rapidly, a large voltage exceeding a tolerable limit may form in a cell membrane and the cell may be significantly damaged. Therefore because of the risk of electrically harming cells, applying a large electric current to an electrode in the beginning should generally be avoided.

When performing electroporation for the first time, an electric current having the same magnitude may be applied to a plurality of electrodes. For each electrode, a potential vₙ of a cell may be sensed to determine a required time τₙ for electroporation to occur. The time taken for the potential vₙ of a cell to reach a preset threshold potential after the electric current is applied may be determined to be the required time τₙ.

Referring to FIG. 4, the required time τₙ for electroporation to occur may be determined to be a different time for each electrode. Since intracellular recording is performed only after electroporation occurs in the cells of all electrodes, when there are different required times τₙ determined for each electrode, there may be a delay in obtaining results of the intracellular recording. That is, even when electroporation occurs quickly in one cell, an electric current may continue to be applied to another cell until electroporation occurs which may cause cell damage. In order to effectively synchronize the various times at which electroporation in the plurality of cells occurs, a device may update, based on the required time τₙ, a magnitude of an electric current to be applied when performing subsequent electroporation for a respective cell.

In an example, when the required time τₙ is greater than a reference time τ_{fixed}, the device may increase the magnitude of the electric current to be applied to a corresponding electrode n so that electroporation may occur more quickly when performing the subsequent electroporation. In addition, when the required time τₙ is less than the reference time τ_{fixed}, the device may reduce the magnitude of the electric current to be applied to the electrode n so that electroporation may occur later when performing the subsequent electroporation.

In an example, a magnitude iₙ of an electric current to be applied to the electrode n may be updated in a time ratio according to Equation 1 below.

$${i}_{n} ← min [ {i}_{n} \cdot {( {τ}_{n} / {τ}_{fixed} )}^{α} , {i}_{MAX} ] \qquad Equation l$$

In Equation 1, α denotes a first proportional regulator and may reflect a correlation (e.g., an inverse propensity) between the required time τₙ and the magnitude iₙ of the electric current determined according to various factors such as i) characteristics of the cell and ii) the distance between the electrode and the cell and/or the arrangement structure. In an example, i_{MAX} may be an electric current set to prevent a large electric current from being applied that may cause damage to the cell. In addition, · indicates multiplication.

In an example, the magnitude iₙ of the electric current to be applied to the electrode n may be updated according to a difference between the required time τₙ and the reference time τ_{fixed} according to Equation 2 below.

$${i}_{n} ← max [ min [ {i}_{n} + β \cdot ( {τ}_{n} - {τ}_{fixed} ) , {i}_{M A X} ] , 0 ] \qquad Equation 2$$

In Equation 2, β denotes a second proportional regulator intended to reflect the correlation (e.g., an inverse propensity) between the required time τₙ and the magnitude iₙ of the electric current determined according to various factors such as i) the characteristics of the cell and ii) the distance between the electrode and the cell and/or the arrangement structure. In an example, i_{MAX} may be an electric current set to prevent a large electric current from being applied that may cause damage to the cell. In Equation 2, max [] may prevent the current in from being updated to a negative value.

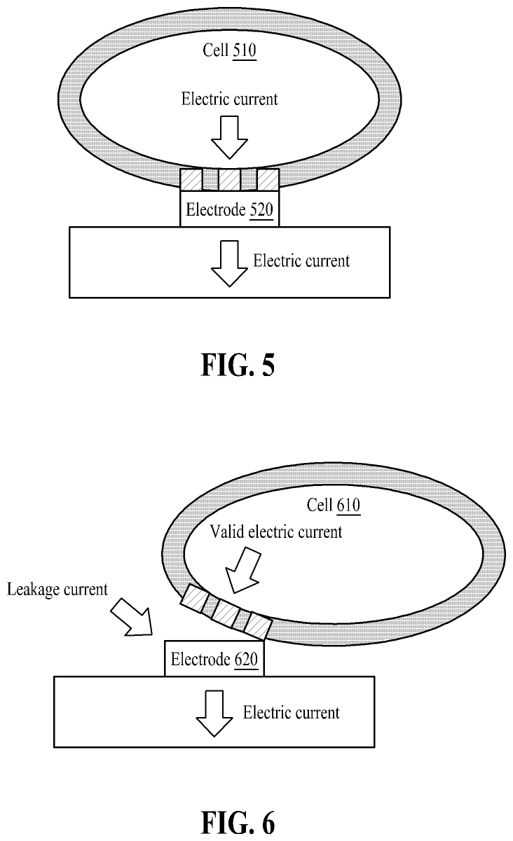

FIGS. 5 and 6 illustrate examples of times required for electroporation to occur according to the location of cells cultured on an electrode according to one or more embodiments.

Referring to FIG. 5, an example of a cell 510 cultured in close contact with an electrode 520 is shown. Since the cell 510 and the electrode 520 are disposed in close contact with each other, all or most of an electric current applied to the electrode 520 may pass through the cell 510 and a cell membrane voltage may be formed that is greater than in the case of FIG. 6 as discussed in greater detail below.

Referring to FIG. 6, an example of a cell 610 cultured not in close contact with an electrode 620 but being spaced apart from the electrode 620 by a certain distance is shown. Since the cell 610 and the electrode 620 are not in close contact with each other, a leakage current may occur between the cell 610 and the electrode 620. Consequently, as a valid electric current that actually flows into the cell 610 may be determined by subtracting the leakage current from an electric current applied to the electrode 620, a resulting cell membrane voltage may be less than in the case of FIG. 5 as discussed above.

Accordingly, if electric currents having the same magnitude were to be applied to both the electrode 520 and the electrode 620, electroporation may occur more rapidly in the cell 510 than in the cell 610. In order to synchronize the time of an occurrence of electroporation in the cell 510 and the cell 610, a larger electric current may be applied to the electrode 620 than to the electrode 520. The larger current may account for, for example, the leakage current that resulted from the space between the electrode 620 and cell 610.

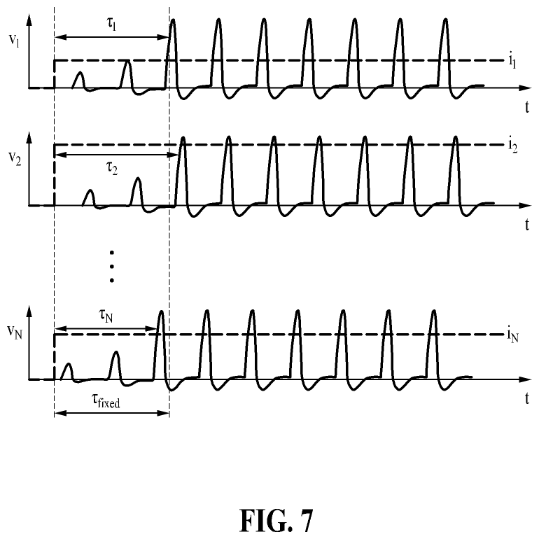

FIG. 7 illustrates an example operation of subsequent electroporation according to one or more embodiments.

Referring to FIG. 7, a potential that is received by each of a plurality of electrodes when an electric current of a previously updated magnitude is applied to each electrode is shown.

The electric current of the magnitude updated in an operation of previous electroporation may be applied to each electrode. For each electrode, the potential vₙ of a cell may be sensed (i.e., received) to determine (i.e., by the processor) the required time τₙ for electroporation to occur. The time taken for the potential vₙ of a cell to reach a preset threshold potential after an electric current is applied may be determined to be the required time τₙ.

Referring to FIG. 7, the required time τₙ for electroporation to occur for each electrode is now similar to each other compared to the example discussed above in FIG. 4. In order to make the time of the occurrence of electroporation in a plurality of cells more uniform, a device may update, based on the required time τₙ, a magnitude of an electric current to be applied when performing subsequent electroporation. For example, when the required time τₙ is greater than the reference time τ_{fixed} for which electroporation is intended to occur, the device may increase the magnitude of the electric current to be applied to a corresponding electrode n so that electroporation may occur more quickly when performing the subsequent electroporation. In addition, when the required time τₙ is less than the reference time τ_{fixed}, the device may reduce the magnitude of the electric current to be applied to the electrode n so that electroporation may occur later when performing the subsequent electroporation. As the description above with reference to FIG. 4 may apply to an operation of updating a magnitude of an electric current to be applied to a corresponding electrode n, a more detailed description is omitted herein.

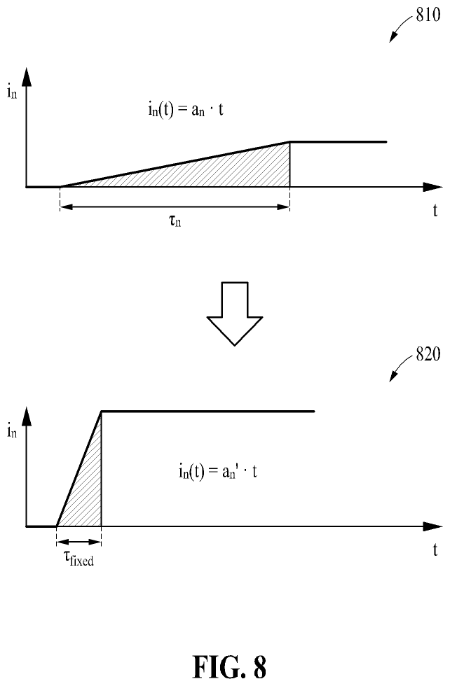

FIG. 8 illustrates an example operation of generating electroporation by increasing the magnitude of an electric current applied to a cell and of performing intracellular recording according to one or more embodiments.

Referring to FIG. 8, an example of where a magnitude of an electric current that is applied to the cell through an electrode changes over time is illustrated. For example, the applied electric current may be configured to increase over time.

As described above with reference to FIGS. 3 to 7, when an electric current of the same magnitude is applied, a considerable amount of time may be required for electroporation to occur for cells that are subject to high leakage currents which may delay the obtaining of a result of intracellular recording. In addition, cell damage may occur in instances where the electric current is continued to be applied to a cell in which electroporation has already occurred. To prevent damage to the cell, the applied electric current may be configured to increase over time. However, increasing the applied electric current should be stopped when electroporation occurs to prevent unnecessary cell damage.

Because the time required for electroporation to occur tends to be inversely proportional to the voltage and inversely proportional to the electric current, when the magnitude of the electric current is constant, a product of the required time and the electric current (i.e., an amount of electric charge Q, which corresponds to an integral value of the electric current for the required time), may be approximately constant.

$$Q = {\int }_{τ} i dt \qquad Equation 3$$

Referring to equation 3, in the case of an electric current defined by

iₙ(t)=a_{fixed}·t

increasing over time, aₙ increase rate an, which may make a Q value for the current required time τₙ to be the same as a Q value for the reference time τ_{fixed} for which electroporation is intended to occur, may be updated according to Equation 4 below.

$${a}_{n} ← {a}_{n} \cdot {( {τ}_{n} / {τ}_{fixed} )}^{α} \qquad Equation 4$$

In Equation 4 above, a denotes a first proportional regulator and may be intended to reflect a correlation (e.g., an inverse propensity) between the required time τₙ and the magnitude iₙ of the electric current determined according to various factors such as i) characteristics of the cell and ii) the distance between the electrode and the cell and/or the arrangement structure.

In an example, the increase rate aₙ of the applied current may be updated according to a difference between the required time τₙ and the reference time τ_{fixed} according to Equation 5 below.

$${a}_{n} ← {a}_{n} + β \cdot ( {τ}_{n} - {τ}_{fixed} ) \qquad Equation 5$$

In Equation 5 above, β denotes a second proportional regulator and may reflect the correlation (e.g., an inverse propensity) between the required time τₙ and the magnitude iₙ of the electric current determined according to various factors such as i) the characteristics of the cell and ii) the distance between the electrode and the cell and/or the arrangement structure.

Referring to FIG. 4, as discussed in greater detail above, a device may measure the current required time τₙ and may subsequently update the increase rate aₙ repeatedly so that electroporation may occur at the reference time τ_{fixed}. For example, as discussed above with respect to FIG. 8, when the required time τₙ of a current operation 810 is greater than the reference time τ_{fixed}, an increase rate aₙ′ of a next operation 820 may be determined to be greater than the increase rate aₙ of the current operation 810. On the other hand, when the required time τₙ of the current operation 810 is less than the reference time τ_{fixed}, the increase rate aₙ′ of the next operation 820 may be determined to be less than the increase rate aₙ of the current operation 810.

For ease of description, FIG. 8 shows a case in which the magnitude of the electric current increases in proportion to time, but the form of increase is not limited thereto, and various forms of electric current increasing over time may be applied without restriction. For example, the magnitude of the electric current may be proportional to the n-th power of time.

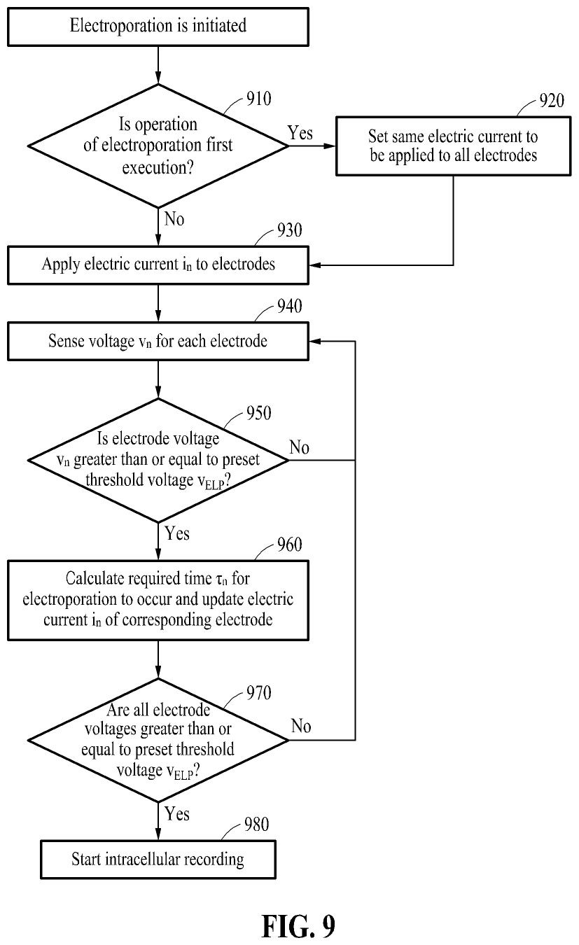

FIG. 9 illustrates an example operation of generating electroporation by applying an electric current and of performing intracellular recording according to one or more embodiments.

In an example, the illustrated operations may be performed sequentially, but not necessarily. That is, in an example, the order of the operations may be changed, and at least two of the operations may be performed in parallel. Operations 910 and 920, which may be performed after electroporation is initiated, may be performed by at least one component (e.g., a processor) of a device.

In an example, the device may determine whether a corresponding operation of electroporation is a first execution in operation 910. When the corresponding operation of electroporation is the first execution, operation 920 may subsequently be performed, and when the corresponding electroporation is not the first execution, operation 930 may subsequently be performed.

In operation 920, the device may set the electric currents to be the same magnitude, the electric current being applied to all electrodes. For example, the device may determine whether to apply an electric current that has the same magnitude or a magnitude that increase over time to a plurality of electrodes.

In operation 930, the device may apply the electric current iₙ to each of the plurality of electrodes. When an electroporation operation is an initial execution, the same electric current determined in operation 920 may be applied to each of the plurality of electrodes. When the electroporation operation is not an initial execution, the previously updated electric current iₙ may be applied to each of the plurality of electrodes. Here, n indicates a natural number from 1 to N, and N may indicate the number of the plurality of electrodes.

In operation 940, the device may sense a voltage vₙ for each electrode. For example, the device may sense a potential of a cell adjacent to each electrode.

In operation 950, the device may determine whether the electrode voltage vₙ is greater than or equal to the preset threshold voltage v_{ELP}. For example, the device may determine whether the potential of the cell sensed in operation 940 is greater than or equal to a preset threshold potential v_{ELP}. When the electrode voltage vₙ is greater than or equal to the preset threshold voltage v_{ELP}, operation 960 may subsequently be performed. On the contrary, when the electrode voltage vₙ is less than the preset threshold voltage v_{ELP}, operation 940 may subsequently be performed. In operation 940, as the electric current may be continuously applied to a corresponding electrode n, the corresponding electrode voltage vₙ may gradually increase.

In operation 960, the device may calculate the required time τₙ for the electroporation to occur and may update the electric current iₙ of the corresponding electrode n based on the required time τₙ. As the above description may apply to an operation of updating the electric current iₙ, a more detailed description is omitted herein.

In operation 970, the device may determine whether all electrode voltages are greater than or equal to a preset threshold voltage v_{ELP}. When some electrode voltages are less than the threshold voltage v_{ELP}, it may be determined that electroporation has not occurred in all electrodes and operation 940 may subsequently be performed. When all electrode voltages are greater than or equal to the threshold voltage v_{ELP}, it may be determined that electroporation has occurred in all electrodes and operation 980 may subsequently be performed.

In operation 980, the device may perform intracellular recording on cells.

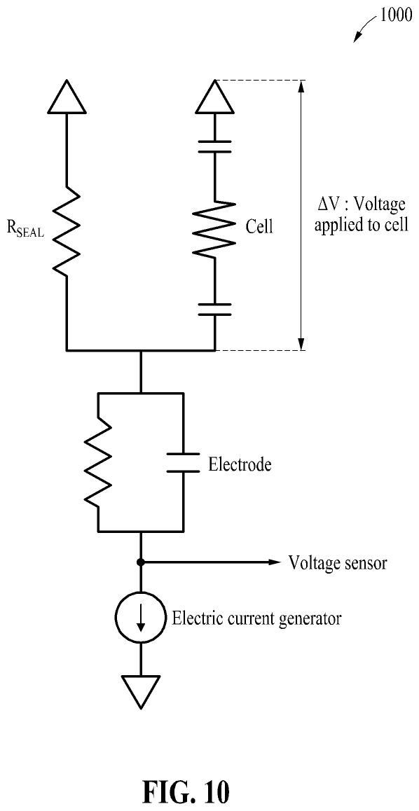

FIGS. 10 to 12 illustrate example operations of determining times required for electroporation to occur, based on a direct current (DC) voltage of a cell according to one or more embodiments.

In FIGS. 3 to 9, the occurrence of electroporation is determined based on a potential of a cell, which is an AC component value. An operation of determining the occurrence of electroporation based on a DC voltage is described in detail with reference to FIGS. 10 to 12.

Referring to FIG. 10, in a non-limiting example, a circuit 1000, which is a DC equivalent to the AC device 200 of FIG. 2, for example, may include a cell, an electrode, a voltage sensor, and an electric current generator. Circuit 1000 is illustrated as a circuit with resistive and current equivalents of the cell, the electrode, leakage current, voltage sensor, and electric current generator. A leakage current may be illustrated as flowing through R_{SEAL} and a voltage applied to the cell may be illustrated as ΔV.

Electroporation tends to occur in a cell when a DC voltage applied to the cell itself increases above a certain level. Using this tendency, the occurrence of electroporation may be determined based on the DC voltage of the cell.

When a position of a cultured cell closest to an electrode is spaced apart from the electrode or the cell is disposed at an angle to the electrode, a leakage current may be generated. Thus, an electric current obtained by subtracting the leakage current from an electric current applied to the electrode may be determined to be a valid electric current that is actually applied to the cell. An applied voltage of the cell may be determined by the valid electric current. When the electric current applied to the electrode is increased, a DC voltage of the cell itself disposed in parallel with the R_{SEAL} may increase and thus, the occurrence of electroporation may be determined based on the DC voltage of the cell itself.

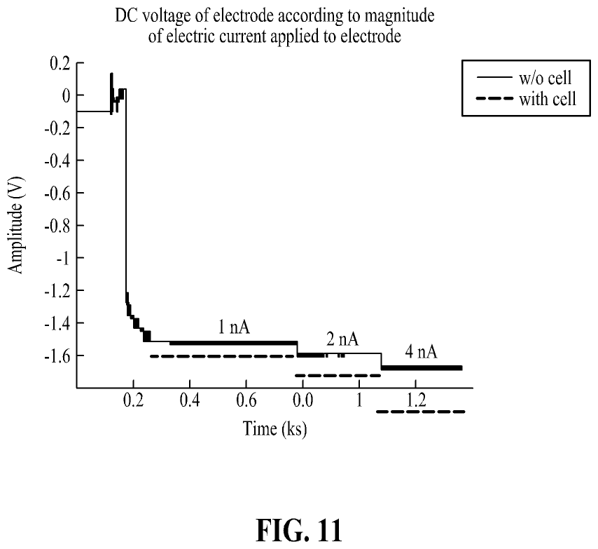

Referring to FIG. 11, a DC voltage of an electrode in a state in which a cell is not cultured is shown as a solid line, and a DC voltage of an electrode in a state in which a cell is cultured is shown as a dotted line. Referring to FIG. 10, since the current flows in the direction from the cell to the electrode, the DC voltage may have a negative value. Referring to FIG. 11, it may be confirmed that the electrode is subjected to a greater voltage when a cell is cultured than when a cell is not cultured.

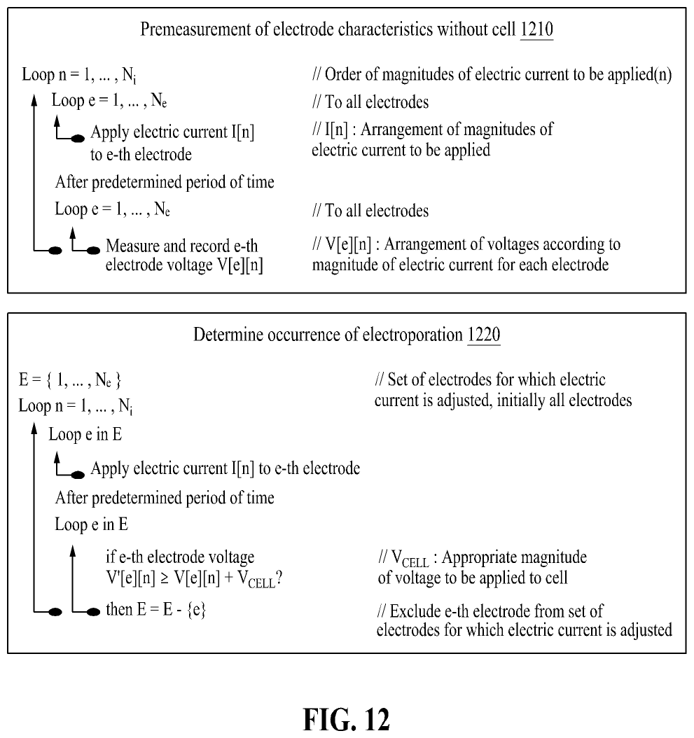

Referring to FIG. 12, an example is shown illustrating an operation of determining whether electroporation has occurred in a cell based on the DC voltage of the cell.

In an example, in operation 1210, the DC voltage of the electrode may be measured when a cell is not cultured. The DC voltage of the electrode measured without a cell may be a criterion for determining the quantity of DC voltage applied to a cell, which may substantially affect electroporation after the cell is cultured. FIG. 12 shows an example in which the number of a plurality of electrodes is Nₑ and the number of magnitudes of an electric current that may be applied is Nᵢ.

For each electrode, a voltage V[e][n] may be measured and recorded a predetermined period of time after an electric current I[1] of a least magnitude to an electric current I[Nᵢ] of a greatest magnitude have been applied.

After a cell is cultured, in operation 1220, a voltage V′[e][n] may be measured and recorded for each electrode when the electric current I[1] of the least magnitude to the electric current I[Nᵢ] of the greatest magnitude have been applied. When a difference between the voltage V′[e][n] and the voltage V[e][n] is less than a predetermined threshold voltage V_{CELL}, the magnitude of the electric current applied to the corresponding electrode may gradually increase. When the difference between the voltage V′[e][n] and the voltage V[e][n] is greater than or equal to the predetermined threshold voltage V_{CELL}, it may be determined that electroporation has occurred in a cell associated with (i.e., cultured on) the corresponding electrode and the electric current applied to the corresponding electrode may not increase. The threshold voltage V_{CELL} may be experimentally determined based on a substantial DC voltage that is applied to the cell when previous electroporation occurred.

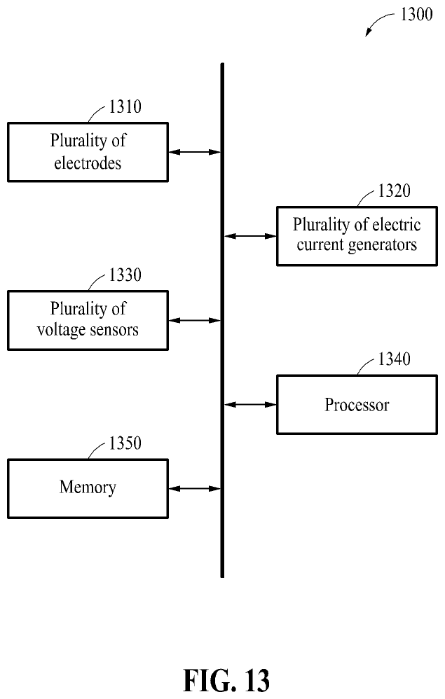

FIG. 13 illustrates an example electronic device for measuring an electrical signal of a cell according to one or more embodiments.

Referring to FIG. 13, in a non-limiting example, an electronic device 1300 for measuring an electrical signal of a cell may include a plurality of electrodes 1310, a plurality of electric current generators 1320, a plurality of voltage sensors 1330, and a processor 1340. In addition, the device 1300 may further include a memory 1350 for storing instructions executable by the processor 1340.

The plurality of electrodes 1310 may come into contact with a medium for culturing cells.

Each of the plurality of electric current generators 1320 may be electrically connected to each of the plurality of electrodes 1310 and may apply an electric current to a cell cultured on a corresponding electrode.

Each of the plurality of voltage sensors 1330 may be electrically connected to each of the plurality of electrodes 1310 and may measure a potential of the cell cultured on the corresponding electrode.

The processor 1340 may be configured to execute programs or applications to configure the processor 1340 to control the electronic apparatus 1300 to perform one or more or all operations and/or methods involving the reconstruction of images, and may include any one or a combination of two or more of, for example, a central processing unit (CPU), a graphic processing unit (GPU), a neural processing unit (NPU) and tensor processing units (TPUs), but is not limited to the above-described examples.

The memory 1350 may include computer-readable instructions. The processor 1340 may be configured to execute computer-readable instructions, such as those stored in the memory 1350, and through execution of the computer-readable instructions, the processor 1340 is configured to perform one or more, or any combination, of the operations and/or methods described herein. The memory 1350 may be a volatile or nonvolatile memory.

In an example, the processor 1340 may determine a required time for electroporation to occur in a target cell based on a potential of the target cell that occurs when an electric current is applied to the target cell cultured on a target electrode among the plurality of electrodes 1310 and may update a magnitude of an electric current to be applied to the target cell based on the required time.

The electronic device 1300 may process the operations described above and a more detailed description thereof is omitted herein.

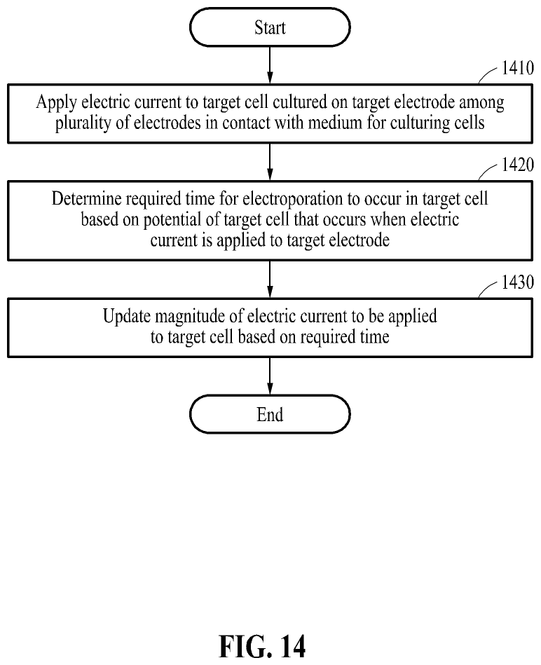

FIG. 14 illustrates an example operating method of a device for measuring an electrical signal of a cell according to one or more embodiments.

In the following example, operations may be performed sequentially, but not necessarily. For example, the order of the operations may be changed, and at least two of the operations may be performed in parallel. Operations 1410 to 1430, which may be performed after electroporation is initiated, may be performed by at least one component (e.g., a processor) of an electronic device (i.e., device 200 of FIG. 2 or electronic device 1300).

Referring to FIG. 14, in a non-limiting example, in operation 1410, the device may apply an electric current to a target cell cultured on a target electrode among a plurality of electrodes in contact with a medium for culturing cells.

In operation 1420, the device may determine a required time for electroporation to occur in the target cell based on a potential of the target cell that occurs when an electric current is applied to the target electrode.

In operation 1430, the device may update a magnitude of an electric current to be applied to the target cell based on the required time.

As the description above with reference to FIGS. 1 to 11 may be applied to each of the operations shown in FIG. 14, a more detailed description is omitted herein.

The device 200, device 300, electronic device 1300, electrodes 1310, electric current generators 1320, voltage sensors 1330, processor 1340, memory 1350, circuit 250, electrodes 240, electric current generators 250, processor 270, circuit 350, electrodes 340, electric current generators 350, processor 370, electrode 520, electrode 620, and circuit 1000 described herein and disclosed herein described with respect to FIGS. 1-14 are implemented by or representative of hardware components. As described above, or in addition to the descriptions above, examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. As described above, or in addition to the descriptions above, example hardware components may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 1-14 that perform the operations described in this application are performed by computing hardware, for example, by one or more processors or computers, implemented as described above implementing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions herein, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media, and thus, not a signal per se. As described above, or in addition to the descriptions above, examples of a non-transitory computer-readable storage medium include one or more of any of read-only memory (ROM), random-access programmable read only memory (PROM), electrically erasable programmable read-only memory (EEPROM), random-access memory (RAM), dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and/or any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above and all drawing disclosures, the scope of the disclosure is also inclusive of the claims and their equivalents, i.e., all variations within the scope of the claims and their equivalents are to be construed as being included in the disclosure.

What is claimed is:

1. A device, comprising:
a plurality of electrodes in contact with a medium for culturing cells;
a plurality of electric current generators, the plurality of electric current generators being electrically and respectively connected to each of the plurality of electrodes and configured to apply an electric current to a cell cultured on a corresponding electrode;
a plurality of voltage sensors, the plurality of voltage sensors being electrically and respectively connected to each of the plurality of electrodes and configured to measure a potential of the cell cultured on the corresponding electrode;
a processor configured to execute instructions; and
a memory storing the instructions, wherein execution of the instructions configures the processor to:
determine a required time for electroporation to occur in a target cell based on a potential of the target cell that occurs in response to an electric current being applied to the target cell cultured on a target electrode among the plurality of electrodes; and
update a magnitude of an updated electric current to be applied to the target cell based on the required time.

2. The device of claim 1, wherein the processor is further configured to:
determine a time taken for a magnitude of the potential of the target cell, in response to the electric current being applied to the target cell, to reach a preset threshold potential to be the required time.

3. The device of claim 1, wherein the processor is further configured to:
in response to the required time being greater than a reference time for which the electroporation is intended to occur, increase the magnitude of the updated electric current; and
in response to the required time being less than the reference time, decrease the magnitude of the updated electric current.

4. The device of claim 1, wherein the processor is further configured to:
determine the required time for electroporation to occur in the target cell while increasing the magnitude of the electric current being applied to the target cell; and
determine a rate of increasing the updated electric current based on the required time.

5. The device of claim 4, wherein the processor is further configured to:
determine the rate of increasing the updated electric current according to an amount of electric charge being applied to the target cell during a reference time for which the electroporation is intended to occur.

6. The device of claim 1, wherein the processor is further configured to:
determine the required time for electroporation to occur in the target cell when the electric current being applied to the target cell is based on a direct current (DC) voltage being applied to the target electrode.

7. The device of claim 6, wherein the processor is further configured to:
in response to a difference between the DC voltage of the target cell and a reference voltage being greater than or equal to a preset threshold voltage, determine that electroporation has occurred in the target cell cultured on the target electrode,
wherein the DC voltage is based on the electric current being applied to the target electrode, and
wherein the reference voltage is based on an electric current being applied to a target electrode on which a cell is not cultured.

8. The device of claim 1, wherein the processor is further configured to:
perform intracellular recording on the cells in response to determining that electroporation has occurred in the cells for each of the plurality of electrodes.

9. An operating method of a device, the operating method comprising:
applying an electric current to a target cell cultured on a target electrode among a plurality of electrodes in contact with a medium for culturing cells;
determining a required time for electroporation to occur in the target cell based on a potential of the target cell that occurs in response to the electric current being applied to the target electrode; and
updating, based on the required time, a magnitude of an updated electric current to be applied to the target cell.

10. The operating method of claim 9, wherein the determining of the required time comprises:
determining a time taken for a magnitude of the potential of the target cell to reach a preset threshold potential to be the required time in response to the electric current being applied to the target cell.

11. The operating method of claim 9, wherein the updating of the magnitude of the electric current comprises:
increasing the magnitude of the updated electric current in response to the required time being greater than a reference time for which the electroporation is intended to occur; and
decreasing the magnitude of the updated electric current in response to the required time being less than the reference time.

12. The operating method of claim 9, wherein the determining of the required time comprises:
determining the required time for electroporation to occur in the target cell while increasing the magnitude of the electric current being applied to the target cell, and
wherein the updating of the magnitude of the electric current comprises:
determining an increase rate of the updated electric current based on the required time.

13. The operating method of claim 12, wherein the updating of the magnitude of the electric current comprises:
determining the increase rate of the updated electric current according to an amount of electric charge applied to the target cell during a reference time for which the electroporation is intended to occur.

14. The operating method of claim 9, wherein the determining of the required time comprises:
determining the required time for electroporation to occur in the target cell when the electric current being applied to the target cell is based on a direct current (DC) voltage being applied to the target electrode.

15. The operating method of claim 14, wherein the determining of the required time comprises:
determining, in response to a difference between the DC voltage of the target cell and a reference voltage being greater than or equal to a preset threshold voltage, that electroporation has occurred in the target cell cultured on the target electrode; and
determining a required time taken until electroporation has occurred in the target cell,
wherein the DC voltage is based on the electric current being applied to the target electrode, and
wherein the reference voltage is based on an electric current being applied to the target electrode on which a cell is not cultured.

16. The operating method of claim 9, further comprising:
performing intracellular recording on the cells in response to determining that electroporation has occurred in the cells for each of the plurality of electrodes.

17. A non-transitory computer-readable storage medium storing instructions that, in response to being executed by a processor, cause the processor to perform the method of claim 9.

18. An apparatus, comprising:
a processor configured to execute instructions; and
a memory storing the instructions, wherein execution of the instructions configures the processor to:
measure a potential of a respective cell cultured on a respective electrode, of a plurality of electrodes, by a respective voltage sensor, of a plurality of voltage sensors, occurring in response to an electric current being applied to the respective cell by the respective electrode, the current being by generated by a respective electric current generator, of a plurality of electric current generators;
determine a required time for electroporation to occur in the respective cell based on the potential of the respective cell; and
update a magnitude of the electric current being applied to the target cell based on the required time.

19. The apparatus of claim 18, wherein the processor is further configured to:
synchronize respective times of electroporation among plural cells including the respective cell by adjusting respective plural magnitudes of respective plural electric currents being applied to the plural cells.

20. The apparatus of claim 19, wherein the adjusting comprises:
increasing a respective plural magnitude in response to a respective required time for a cell among the plural cells being greater than a reference time for which the electroporation is intended to occur; and
decreasing the respective plural magnitude in response to the respective required time being less than the reference time.
